(19) United States
(12) Patent Application Publication
(10) Pub. No.: US 2024/0210675 A1
(43) Pub. Date: Jun. 27, 2024

(54) VISION SIMULATION DEVICE FOR PROVIDING A CORRECTION OF VISUAL DEFECTS AND METHOD FOR OPERATING A VISION SIMULATION DEVICE

(71) Applicant: SCHWIND EYE-TECH-SOLUTIONS GMBH, Kleinostheim (DE)

(72) Inventors: Samuel ARBA MOSQUERA, Aschaffenburg (DE); Pascal NAUBEREIT, Laufach (DE)

(21) Appl. No.: 18/390,646

(22) Filed: Dec. 20, 2023

(30) Foreign Application Priority Data
Dec. 21, 2022 (DE) 10 2022 134 292.1

(51) Int. Cl. G02B 27/00

(52) U.S. Cl. CPC G02B 27/0068

(57) ABSTRACT

The invention relates to a vision simulation device (10) for providing a correction of visual defects and to a method for operating the vision simulation device (10). The vision simulation device (10) includes an eye interface (16), at least one spatially controllable light wave modulator (12) and a control device (14), wherein the at least one spatially controllable light wave modulator (12) is arranged and formed to modulate light waves (20) for generating aberrations and to provide the modulated light waves (22′) at the eye interface (16) of the vision simulation device (10), wherein the control device (14) is formed to drive the spatially controllable light wave modulator (12) for at least partially compensating for at least one predetermined visual defect by generating at least one preset aberration.

### FIELD

The invention relates to a vision simulation device for providing a correction of visual defects and to a method for operating such a vision simulation device.

### BACKGROUND

For a visual simulation of refractive corrections, phoropters or also processed PMMA plates, which have been processed with a laser for a patient, can for example be used. Thus, for example with a phoropter, a subjective refraction can be determined and/or a correction can be simulated in that various lenses are held up to a patient, in particular until a lens or a lens combination subjectively provides optimum values for the patient. However, phoropters do not allow providing all of corrections, in particular not of higher order visual defects, whereby they are often insufficiently taken into account.

It is the object of the present invention to ascertain and/or visualize visual defects in improved manner.

### SUMMARY

This object is solved by the independent claims. Advantageous embodiments of the invention are disclosed in the dependent claims, the following description as well as the figures.

The invention is based on the idea that corrections of visual defects, preferably also of higher order, are simulated and visually visualized and made experienceable for a patient by means of one or more light wave modulators, in particular deformable phase plates. Thus, the patient can for example also previously visually experience a sought treatment by an ophthalmological laser on a cornea and/or the patient can subjectively decide when the best correction of the visual defects is present for him based on an adjustment of the light wave modulator, wherein correction data for the visual disorder correction by means of a laser treatment can then be provided from this adjustment.

An aspect of the invention relates to a vision simulation device for providing a correction of visual defects. The vision simulation device includes an eye interface, at least one spatially controllable light wave modulator and a control device, wherein the at least one spatially controllable light wave modulator is arranged and formed to modulate light waves for generating aberrations and to provide the modulated light waves at the eye interface of the simulation device, wherein the control device is formed to drive the spatially controllable light wave modulator for at least partially compensating for at least one predetermined visual defect by generating at least one preset aberration.

In other words, light waves, which can enter an eye of a patient via an eye interface, can be modulated by the spatially controllable light wave modulator or beam modulator to simulate aberrations, in particular higher order aberrations. Therein, a change of spatial phase portions and/or identity portions of light waves, which in particular originate from a display, is meant by modulating. Therein, the light waves can originate from outside of the vision simulation device, in particular from an external display image, and/or be generated by a display device of the vision simulation device. Therein, the light waves are preferably in a visible spectral range from circa 380 nanometers to 760 nanometers and can be monochromatic or polychromatic.

Preferably, the light wave modulator can be controlled by a control device such that a predetermined visual defect, which was for example determined by means of preceding diagnostic measurement, is compensated for by the generated aberration. This means, the generated aberration can be a planned correction, which compensates for the predetermined visual defect. Alternatively, aberrations can for example also be specifically induced, wherein visual defects can be identified for a treatment for the patient by a subjective patient feedback. Therein, the control device can be formed as a computer or processor, in particular microprocessor, wherein the control device can be formed to execute a program code, whereby a control of the spatially controllable light wave modulator is effected.

By a spatially controllable light wave modulator, it is meant that various points of the light waves, in particular viewed in a cross-section, can be driven and changed such that a modulation arises for the respective position in cross-section. This means that spatially arranged elements of the light wave modulator, for example in a surface or a volume, can be separately driven such that a characteristic of these elements, in particular a position and/or orientation, is changed. For example, the light wave modulator can be formed by means of mirrors, liquid crystals, gratings, prisms and/or phase plates. Thus, orientations and/or characteristics of the previously mentioned components can for example change by drive of the light wave modulator, such as for example an orientation of liquid crystals, a position and/or inclination angle of mirrors and further. Thus, it is achieved that various portions of the light waves travel different optical path distances in the light wave modulator, whereby a phase is for example modulated and thus aberrations are induced.

Aberrations, which are generated by the spatially controllable light wave modulator, can include aberrations from the zeroth order, wherein higher order aberrations can preferably be generated, such as for example coma, spherical aberrations, trefoil and further. By means of the generated aberrations, visual defects, in particular predetermined visual defects of a patient, which can for example be provided by means of Zernike polynomials, Gatinel polynomials, Fourier series and/or a correction matrix, can then be visualized for a patient and/or be at least partially compensated for. By at least partially compensating for, it is meant that only one of multiple visual defects can for example also be compensated for and/or that a visual defect is only compensated for up to a certain magnitude, wherein all of the visual defects are preferably completely compensated for. Aberrations can for example also be deliberately induced, which simulate multifocality and presbyopia correction, respectively, and/or a noise signal can be added to generate a more realistic vision estimation for a patient after a potential correction/laser correction.

The advantage arises by the vision simulation device that an imagination can be achieved for the patient before a treatment by a visual simulation and thus a decision aid for or against a certain treatment can be made. On the other hand, corrections can be better adapted to actual or subjective needs of a patient. Thus, an adaptation of a correction of visual defects and/or of a noise signal can for example be manually, automatically or semi-automatically made based on a feedback from the patient.

The invention also includes embodiments, by which additional advantages arise.

An embodiment provides that the spatially controllable light wave modulator is formed as a wavefront modulator. Wavefront modulators are actively controllable devices, which are formed to locally change optical path lengths of the light, which in particular include a geometric length and/or a refractive index on this path. Herein, reflective or transmissive wavefront modulators can be employed, which for example include deformable mirrors and/or liquid crystals. In so-called spatial liquid crystal light modulators (Liquid Chrystal Spatial Light Modulators; LC-SLM), densely packed LC cells are provided, which change an orientation by driving by means of a voltage signal to locally adapt the optical path length. Thus, a high spatial resolution can be generated, in particular with discrete phase shifts or continuous progression. With deformable mirrors, an incoming light wave is reflected on the mirrors, wherein the mirrors can be spatially departed or approached to adapt an optical path length of the various spatial portions of the light wave. Hereby, preferred formations of the spatially controllable light wave modulator can be provided.

In a particularly preferred embodiment, it is provided that the spatially controllable light wave modulator is formed as a deformable phase plate. With a deformable phase plate, a wavefront is modulated by a transmissive passage through the phase plate. Therein, the phase plate comprises a liquid with high refractive index, which is enclosed in a membrane. The membrane can be spatially selectively shifted or distorted by applying a voltage signal, whereby different optical path lengths through the liquid with high refractive index arise for the respectively arriving and spatially distributed light waves. The formation of the light wave modulator as a deformable phase plate has the advantage that it can be transmissively used and thus little changes have to be performed in a beam path of the vision simulation device. A deformable phase plate also only needs little installation space and provides a high resolution.

A further embodiment provides that the eye interface is binocularly formed and respective light wave modulators spatially controllable separately from each other are provided for the respective binoculars. This means that a patient can look through the eye interface with both eyes at the same time, wherein aberrations for providing a correction of visual defects can be achieved separately for each eye. Instead of providing two light wave modulators controllable separately from each other, only one increased light wave modulator can alternatively also be provided, for example an extended light wave modulator, which modulates light waves, which for example were previously divided, for the respective binoculars.

A further embodiment provides that multiple respectively spatially controllable light wave modulators are arranged one behind the other. Thus, a range of a phase change can in particular be increased and/or achromatic effects, in particular achromatic doublets and/or a triplet achromat can be simulated.

Preferably, it is provided that the control device is formed to generate modulation data for the spatially controllable light wave modulator from predetermined visual disorder data, which includes information about visual defects of a patient, and to drive the spatially controllable light wave modulator by means of the modulation data to generate an aberration, in particular higher order aberrations, in the light waves, which at least partially compensate for the visual defect. In other words, a planned correction of the visual disorder can be examined by the vision simulation device. Hereto, the vision simulation device can for example be coupled to a diagnostic appliance or a planning station, wherein the diagnostic appliance ascertains the visual disorder data. The correction ascertained therefrom can then be directly displayed to the patient via the vision simulation device. For example, Zernike coefficients, Gatinel-Malet coefficients, a correction matrix or 2D or 3D Fourier series can respectively be provided for a preset pupil size and optionally preset centering, which the control device converts into modulation data for driving the control modulator. Hereby, the advantage arises that an effect of a planned correction can be visualized already before a treatment, whereby a success of the planned treatment can be examined.

Preferably, it is provided that the control device is formed to specifically induce aberrations for the spatially controllable light wave modulator and to provide an adjustment of the spatially controllable light wave modulator for providing a subjective visual defect correction depending on a subjective patient feedback. Hereto, the vision simulation device can for example comprise an input device, which is formed to capture the subjective patient feedback, wherein the adjustments of the light wave modulator are stored if a positive feedback is captured. The adjustment, which provides the subjective visual defect, can then for example be used to perform a glasses correction and/or generate control data for an ophthalmological laser, which corrects the visual defect. Thus, a diagnosed profile can for example be adapted according to patient preferences or according to a subject vision perception, in particular in case of complex visual defects.

A further embodiment provides that the vision simulation device further comprises one or more lenses, in particular with different diopter values and/or designs, which can be introduced into a beam path of the light waves. In other words, the vision simulation device can be combined with a phoropter to ascertain a refraction, which is provided by the lenses of the phoropter, in addition to the aberrations, in particular higher order aberrations, which are generated by the light wave modulator. Preferably, they can be movable lenses, which can be introduced into and removed from the beam path. For example, the lenses can have a spherical and/or cylindrical design, in particular with different cylinder axes. The lenses can also be formed in analogy to a Badal optometer or as Alvarez-Lohman lenses.

A further embodiment provides that the vision simulation device further comprises an eye tracking device, which is formed to ascertain a positioning and/or viewing direction of an eye located at the eye interface, wherein the control device is further formed to drive the spatially controllable light wave modulator depending on the ascertained position and/or viewing direction. This means that one or more cameras can for example be provided as the eye tracking device, which track the positioning and/or the viewing direction of the eye or eyes. In other words, the eye tracking device can be an eye tracking system. Thus, the control device can adequately modulate the light waves according to position and/or a viewing direction of the eye to generate the suitable aberrations. For example, spherical aberrations can thus be induced and compensated for, respectively, in improved manner.

A further aspect of the invention relates to a method for operating the previously mentioned vision simulation device, wherein visual disorder data of an eye of a patient is determined by a diagnostic device and a correction of visual defects is ascertained from the visual disorder data, wherein modulation data for the at least one spatially controllable light wave modulator is generated by the control device of the vision simulation device, by which the correction of the visual defects is simulated in the vision simulation device, wherein the at least one spatially controllable light wave modulator is driven by means of the modulation data for generating aberrations, which provide the correction of the visual defects. Herein, the same advantages and possibilities of variation as in the vision simulation device arise.

The method can include at least one additional step, which is executed if and only if an application case or an application situation occurs, which has not been explicitly described here. For example, the step can include the output of an error message and/or the output of a request for inputting a user feedback. Additionally or alternatively, it can be provided that a default setting and/or a predetermined initial state are adjusted.

The control device can be formed to perform the steps of at least one embodiment of the previously described method. Thereto, the control device can comprise a computing unit for electronic data processing such as for example a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which the steps of the respective embodiment of the respective method are encoded. The program code can include the control data for the respective laser. The program code can be executed by means of the computing unit, whereby the control device is caused to execute the respective embodiment. The control device can be formed as a control chip or control unit. The control device can for example be encompassed by a computer or computer network.

Further features and advantages of one of the described aspects of the invention can result from the embodiments of the other aspect of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following can be present in any combination with each other and/or the features of the embodiments. This means, the features of the execution examples can supplement and/or replace the features of the embodiments and vice versa. Thus, configurations are also to be regarded as encompassed and disclosed by the invention, which are not explicitly shown or explained in the figures, but arise from and can be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims.

FIG. 1 depicts a schematically illustrated vision simulation device according to an exemplary embodiment.

FIG. 2 depicts a schematically illustrated vision simulation device according to an exemplary embodiment.

FIG. 3 depicts a schematic method diagram according to an exemplary embodiment.

In the figures, identical or functionally identical elements are provided with the same reference characters.

### DETAILED DESCRIPTION

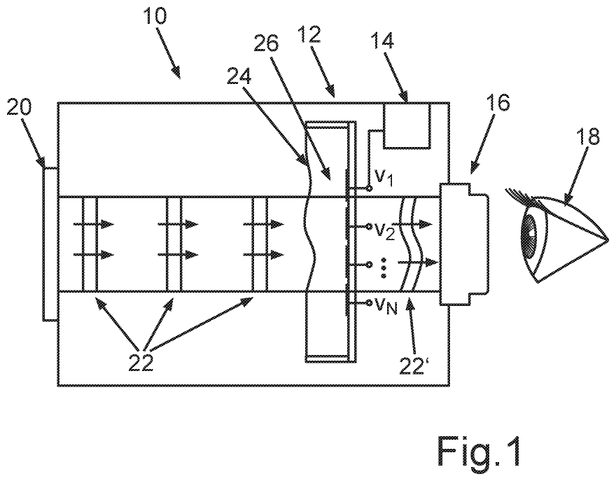

In FIG. 1, a schematic side view of a vision simulation device 10 according to an exemplary embodiment is illustrated. The vision simulation device 10 can include at least one spatially controllable light wave modulator 12, a control device 14 for controlling the light wave modulator 12 and an eye interface 16, by which an eye 18 can look into or through the vision simulation device 10. Furthermore, a display device 20 can be provided, which can belong to the vision simulation device 10 or is external thereto, wherein the display device 20 can provide light waves 22, for example of a display image. Therein, the light waves 22 are preferably monochromatic or polychromatic and have a wavelength in a visible spectral range from 380 nanometers to 760 nanometers. Alternatively to the display device 20 or additionally, the light waves 22 can originate from another source, for example from a room behind the vision simulation device 10.

The spatially controllable light wave modulator 12 can be formed as a reflective or transmissive wavefront modulator, wherein the spatially controllable light wave modulator 12 is formed as a deformable phase plate in this embodiment, which can modulate the light waves 22 by transmission through the deformable phase plate 12. Alternatively or additionally, deformable mirrors or a liquid crystal modulator can for example be used as the light wave modulator 12. The deformable phase plate 12 includes a chamber with refractive liquid 26, wherein the refractive liquid 26 is delimited to one side by a conductive membrane 24. If a voltage V₁, V₂, . . . , V_{N} is applied to a respective spatial area of the deformable phase plate 12 by the control device 14, which can for example be formed as a processor, in particular microprocessor, the membrane 24 can be deformed corresponding to the applied voltage, whereby an optical path length of the light waves 22 through the deformable phase plate 12 and through the refractive liquid 26, respectively, changes. Accordingly, a phase of the light waves 22 is changed and modulated light waves 22′ can be output at the eye interface 16. Thus, aberrations can for example be induced in the light waves 22, in particular higher order aberrations, such as for example coma, spherical aberrations and/or trefoil.

Preferably, the control device 14 can be formed to drive the deformable phase plate 12 such that the aberrations, which are generated in the modulated light waves 22′, completely or partially compensate for one or more visual defects of the eye 18. Thus, visual disorder data of the eye 18 can for example be present for the control device 14, which includes information about visual defects. For example, the visual disorder data can include Zernike polynomials, Gatinel-Malet coefficients, Fourier series or a correction matrix with respective visual defects and aberrations of the eye 18, respectively. The control device 14 can generate modulation data for the deformable phase plate 12 from it, such that the deformable phase plate 12 generates aberrations, which invert these visual defects. Thus, a treatment or planned correction on a cornea of the eye 18 can for example be simulated to examine if the correction provides the desired success.

Alternatively or additionally, the deformable phase plate 12 can be driven by the control device 14 such that aberrations of different magnitudes are specifically provided in the modulated light waves 22′. A patient can then give a subjective feedback whether or not a vision improves with the respective adjusted aberration. Thereto, an input device (not shown) can for example be provided, via which this subjective feedback can be captured and/or via which a further or next adjustment for the deformable phase plate 12 can be generated. If one of these adjustments is satisfactory, the corresponding modulation data can be stored by the control device 14, wherein control data for an ophthalmological laser for a treatment of a cornea of the eye 18 can for example be generated from the modulation data.

Thus, a predetermined correction can be examined and/or correction data for a correction can be ascertained by the vision simulation device 10, in particular before a treatment.

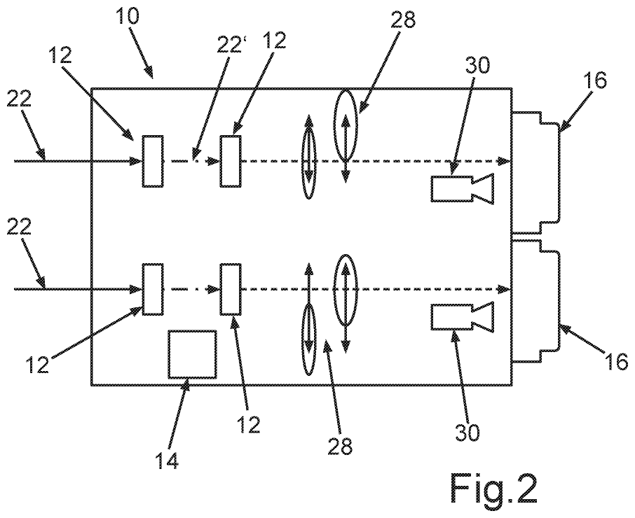

In FIG. 2, a further exemplary embodiment of a vision simulation device 10 is illustrated. Herein, a schematic top view to the vision simulation device 10 is shown, which is binocularly formed in this embodiment. This means that two eye interfaces 16 for a respective eye of a patient are provided. In the binocular configuration, respective beam paths for the light waves 22 can be provided with respective spatially controllable light wave modulators 12, which can in particular be separately driven by the control device 14 for generating different aberrations. Furthermore, multiple spatially controllable light wave modulators 12 can be arranged in a respective beam path, preferably one behind the other (e.g., in series), which can for example generate different aberrations and/or augment a magnitude of a generated aberration.

In the vision simulation device 10, one or more lenses 28, in particular with different power, can also be provided, which can be introduced into a beam path of the light waves 22, 22′. Preferably, the lenses 28 can be mechanically and/or electrically moved into or out of the beam path to also generate refractions, for example spherical and/or cylindrical refractions, in addition to the aberrations, which are generated by the light wave modulators 12.

Furthermore, the vision simulation device 10 can include one or more eye tracking devices 30 or eye trackers, which are formed to ascertain a positioning or viewing direction of a respective eye 18 applied to the eye interface 16. The positioning and/or viewing direction can then be provided to the control device 14, which drives the respective light wave modulator 12 depending thereon to generate adequate aberrations for the position and/or viewing direction of the eye 18.

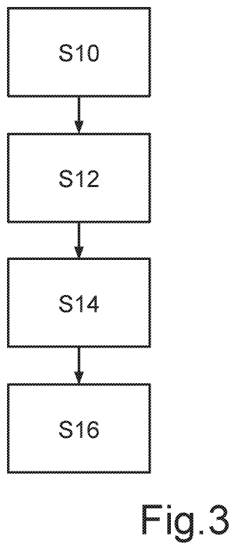

In FIG. 3, a schematic method diagram for operating a vision simulation device 10 according to an exemplary embodiment is illustrated. In a step S10, visual disorder data of an eye 18 can be determined by an external diagnostic device, wherein a planned correction of visual defects can be ascertained from the visual disorder data in a step S12.

In a step S14, the control device 14 of the vision simulation device 10 can generate modulation data for the at least one spatially controllable light wave modulator 12, by which a correction of the ascertained visual defects is simulated in the vision simulation device 10.

In a step S16, the at least one spatially controllable light wave modulator 12 can finally be driven by means of the modulation data for generating aberrations, which provide the correction of the visual defects. Thus, it can be examined if the visual defects have been correctly ascertained from the visual disorder data and/or correction data for an ophthalmological laser can be validated before a treatment occurs.

Overall, the examples show how a visual simulation of a correction of visual defects can be provided for a patient by the invention.

1. A vision simulation device for providing a correction of visual defects, comprising:
an eye interface;
at least one spatially controllable light wave modulator configured to modulate light waves for generating aberrations and to provide the modulated light waves at the eye interface of the vision simulation device; and
a control device configured to drive the at least one spatially controllable light wave modulator for at least partially compensating for at least one predetermined visual defect by generating at least one preset aberration.

2. The vision simulation device according to claim 1, wherein the at least one spatially controllable light wave modulator comprises a wavefront modulator.

3. The vision simulation device according to claim 1, wherein the at least one spatially controllable light wave modulator comprises a deformable phase plate.

4. The vision simulation device according to claim 1, wherein the eye interface is binocularly formed and respective light wave modulators spatially controllable separately from each other are provided for the respective binoculars.

5. The vision simulation device according to claim 1, wherein multiple respectively spatially controllable light wave modulators are arranged one behind another.

6. The vision simulation device according to claim 1, wherein the control device is configured to:
generate modulation data for the at least one spatially controllable light wave modulator from predetermined visual disorder data, wherein the predetermined visual disorder data includes information about visual defects of a patient, and
drive the at least one spatially controllable light wave modulator based on the modulation data to generate an aberration in the light waves to at least partially compensate for the visual defect.

7. The vision simulation device according to claim 1, wherein the control device is configured to:
induce aberrations for the at least one spatially controllable light wave modulator, and
provide an adjustment of the at least one spatially controllable light wave modulator for providing a subjective visual defect correction depending on a subjective patient feedback.

8. The vision simulation device according to claim 1, wherein the vision simulation device is configured to be introduced into a beam path of the light waves.

9. The vision simulation device according to claim 1, wherein the vision simulation device further comprises an eye tracking device configured to ascertain a position and/or viewing direction of an eye located at the eye interface,
wherein the control device is further configured to drive the at least one spatially controllable light wave modulator based on the ascertained position and/or viewing direction.

10. A method for operating a vision simulation device according to claim 1, the method comprising:
determining, via a diagnostic device, visual disorder data of an eye of a patient;
ascertaining a correction of visual defects from the visual disorder data;
generating, via the control device of the vision simulation device, modulation data for the at least one spatially controllable light wave modulator to simulate correction of the visual defects; and
driving the at least one spatially controllable light wave modulator based on the modulation data for generating aberrations to correct the visual defects.

11. The vision simulation device according to claim 6, wherein the generated aberration is a higher order aberration.

12. The vision simulation device according to claim 1, further comprising a second eye interface, wherein the at least one spatially controllable light wave modulator includes a first light wave modulator aligned with the eye interface and a second light wave modular aligned with the second eye interface, wherein the first light wave modulator and the second light wave modulator are spatially controllable separately from each other.
